SEQUENCE LISTING (13 sequences)

SEQ ID NO: 1 - DNA, 25 bp, Artificial Sequence
  Complementary MO to MRJ
cagcatctgctccttaccatttatt

SEQ ID NO: 2 - DNA, 25 bp, Artificial Sequence
  Control MO of MoMRJ
cctcttacctcagttacaatttata

SEQ ID NO: 3 - DNA, 38 bp, Homo sapiens
  primer_bind (1)..(38)
cacttgatggcttacccttatgatgtgccagattatgc

SEQ ID NO: 4 - DNA, 32 bp, Homo sapiens
  primer_bind (1)..(32)
cacttggaattcacctgctgcggacgcgaggg

SEQ ID NO: 5 - DNA, 33 bp, Homo sapiens
  primer_bind (1)..(33)
cacttggagctcgttcctgttaatcctcaaatg

SEQ ID NO: 6 - DNA, 20 bp, Homo sapiens
  primer_bind (1)..(20)
agcacggcatcgtcaccaac

SEQ ID NO: 7 - DNA, 20 bp, Homo sapiens
  primer_bind (1)..(20)
tggctggggtgttgaaggtc

SEQ ID NO: 8 - DNA, 21 bp, respiratory syncytial virus
  primer_bind (1)..(21)
gcaggattgtttatgaatgcc

SEQ ID NO: 9 - DNA, 22 bp, respiratory syncytial virus
  primer_bind (1)..(22)
cttccacaacttgytccatttc

SEQ ID NO: 10 - DNA, 24 bp, respiratory syncytial virus
  primer_bind (1)..(24)
catgagcaaactcctcactgaact

SEQ ID NO: 11 - DNA, 25 bp, respiratory syncytial virus
  primer_bind (1)..(25)
tcttgggtgaatttagctcttcatt

SEQ ID NO: 12 - DNA, 22 bp, respiratory syncytial virus
  primer_bind (1)..(22)
cacaacaatgccagtgctacaa

SEQ ID NO: 13 - DNA, 26 bp, respiratory syncytial virus
  primer_bind (1)..(26)
ttagaccattaggttgagagcaatgt

(19) United States
(12) Patent Application Publication
(10) Pub. No.: US 2019/0330635 A1
(43) Pub. Date: Oct. 31, 2019

(54) ANTIVIRAL AGENT AND METHOD FOR TREATING VIRAL INFECTION

(71) Applicants: Li-Min Huang, Taipei City (TW); Shih-Han Huang, Taipei City (TW)

(72) Inventors: Li-Min Huang, Taipei City (TW); Shih-Han Huang, Taipei City (TW)

(21) Appl. No.: 16/475,168

(22) PCT Filed: Jan. 24, 2018
(86) PCT No.: PCT/IB2018/050421

§ 371 Date: Jul. 1, 2019

Related U.S. Application Data
(60) Provisional application No. 62/449,600, filed on Jan. 24, 2017.

(51) Int. Cl. C12N 15/113; A61P 31/18; A61P 31/14; A61K 31/522; A61K 31/7072; A61K 38/21; A61K 31/655; A61K 31/245

(52) U.S. Cl. CPC C12N 15/1132; A61P 31/18; A61P 31/14; C12N 15/1131; A61K 31/522; C12N 2310/3233; A61K 38/21; A61K 31/655; A61K 31/245; C12N 2310/11; A61K 31/7072

(57) ABSTRACT

The present invention relates to antiviral agents and methods of their use in suppression of viruses and in the treatment of a disease or condition associated with viral infection. The antiviral agent includes a nucleotide derivative is a morpholino oligomer complementary to mammalian relative of DnaJ (MRJ) gene.

### CROSS-REFERENCE TO RELATED APPLICATIONS

This application claims priority to U.S. Provisional Application Ser. No. 62/449,600, filed Jan. 24, 2017, which is herein incorporated by reference in its entirety for all purposes.

### BACKGROUND

### 1. Technical Field

The invention relates to antisense oligonucleotides for use in treating virus infection and antiviral treatment methods employing the oligonucleotides.

### 2. Description of Associated Art

Bacterial and viral infections remain major problems for human health (Morens and Fauci, 2013). Treatment of bacterial infections largely relies on antibiotics (Bassetti et al., 2016; Bush and Bradford, 2016), whereas the mainstay of antiviral therapy remains supportive care and placating the symptoms. Moreover, emerging and re-emerging pathogens continue to plague humans as a consequence of the continued encroachment of civilization on wild areas. The lack of timely available antiviral agents has complicated the management of viral outbreaks, and thus the development of broad-spectrum antiviral strategies is highly desired (Vigant et al., 2015).

At present, only several antiviral agents are available. The current antiviral agents initially act on specific viral gene products, such as human immunodeficiency virus type 1 (HIV-1) protease and reverse transcriptase and hepatitis C non-structural proteins, to interfere with viral replication (O'Connor et al., 2017; Spengler, 2017). Moreover, new antiviral approaches have been developed to target viral-host interactions or cellular components required for viral propagation, such as inhibiting viral entry and fusion with the host plasma membrane or the activity of viral polymerases, or manipulating the host immune response (Brito and Pinney, 2017; Ko et al., 2017; Prasad et al., 2017).

However, there is still an unmet need for a broad-spectrum antiviral agent that can effectively treat viral infection caused by a variety of virus despite the highly mutagenic characteristic of virus.

### SUMMARY

In view of the foregoing, an antiviral agent is provided. The antiviral agent comprises a nucleotide derivative complementary to a mammalian relative of DnaJ (MRJ) gene, wherein the nucleotide derivative comprises at least one nucleotide with a sugar moiety being substituted with morpholine.

In one embodiment of the present disclosure, the nucleotide derivative is a morpholino oligomer. In another embodiment of the present disclosure, the nucleotides in the nucleotide derivative are morpholino nucleotides.

In one embodiment of the present disclosure, the nucleotide derivative in the antiviral agent is complementary to intron 8 of the MRJ gene. In another embodiment of the present disclosure, the nucleotide derivative is complementary to 5′ splice site region of intron 8 of the MRJ gene. In yet another embodiment of the present disclosure, the MRJ gene is human MRJ gene.

In one embodiment of the present disclosure, the nucleotide derivative in the antiviral agent comprises about 20 to about 40 nucleotides. In another embodiment of the present disclosure, the nucleotide derivative is no more than 30 nucleotides in length. In yet another embodiment of the present disclosure, the nucleotide derivative is 25 nucleotides in length.

In one embodiment of the present disclosure, the nucleotide derivative comprises SEQ ID NO: 1. In another embodiment of the present disclosure, the nucleotide derivative may be a sequence of SEQ ID NO: 1, that is, the nucleotide derivative consists of a sequence that is exactly SEQ ID NO: 1 with no additional sequence. In another aspect of the present disclosure, a use of the antiviral agent in treating a disease or a condition associated with viral infection in a subject in need thereof is provided.

In one embodiment of the present disclosure, the viral infection is caused by a virus selected from the group consisting of cytomegalovirus (CMV), Epstein-Barr virus (EBV), human immunodeficiency virus-1 (HIV-1), human immunodeficiency virus-2 (/HIV-2), human metapneumovirus, human parainfluenza virus (HPIV), influenza virus, respiratory syncytial virus (RSV), adenovirus, rhinovirus, coronavirus, enterovirus 71 (EV-71), Enterovirus D68 (EV-D68), coxsackievirus, dengue virus, Japanese encephalitis virus (JEV), and any combination thereof. In another embodiment of the present disclosure, the viral infection is caused by CMV, EBV, HIV, influenza virus, RSV or any combination thereof. In yet another embodiment of the present disclosure, the viral infection is caused by RSV.

In one embodiment of the present disclosure, the disease or the condition associated with viral infection is selected from the group consisting of retinitis (caused by, e.g., CMV), colitis (caused by, e.g., CMV), infectious mononucleosis (caused by, e.g., CMV and EBV), Hodgkin's lymphoma (caused by, e.g., EBV), Burkitt's lymphoma (caused by, e.g., EBV), nasopharyngeal carcinoma (caused by, e.g., EBV), acquired immune deficiency syndrome (AIDS; caused by, e.g., HIV-1 and HIV-2), upper respiratory tract infection (URI), lower respiratory tract infection (LRI; caused by, e.g., HPIV, adenovirus, RSV, coronavirus, rhinovirus, and EV-D68), myocarditis (caused by, e.g., coxsackievirus), encephalitis (caused by, e.g., EV-71, EV-D68, dengue virus, and JEV), dengue hemorrhagic fever and dengue shock syndrome (DHF/DSS; caused by, e.g., dengue virus), and any combination thereof. In another embodiment of the present disclosure, the disease or condition associated with viral infection is URI or LRI.

In another aspect of the present disclosure, a method for suppressing viral infection is provided. The method comprises administering the antiviral agent to a subject in need thereof. In one embodiment of the present disclosure, the viral infection may be caused by CMV, EBV, HIV, influenza virus, RSV or any combination thereof.

In one embodiment of the present disclosure, the method further comprises administering an additional antiviral therapy to the subject. In one embodiment of the present disclosure, the additional antiviral therapy may be selected from the group consisting of carbovir, acyclovir, interferon, stavudine, 3′-azido-2′,3′-dideoxy-5-methyl-cytidine (CS-92), β-D-dioxolane nucleosides, oseltamivir phosphate, and any combination thereof.

In one embodiment of the present disclosure, the method further comprises administering an antibiotic to the subject when the subject has a secondary bacterial infection.

The antiviral agent of the present disclosure is useful in the treatment of viral infection, particularly the infection caused by human RSV, which is a major cause of viral bronchiolitis and pneumonia in infants and the elderly worldwide, and human immunodeficiency virus type 1 (HIV-1).

### BRIEF DESCRIPTION OF THE DRAWINGS

The present disclosure can be more fully understood by reading the following detailed description of the embodiments, with reference made to the accompanying drawings, wherein:

FIG. 1A shows the illustration of MRJ pre-mRNA substrate containing exons 8 and 9 and internally truncated intron 8. The antisense morpholino is complementary to the 5′ splice site of MRJ intron 8 and its binding prevents Ul from acting on the splice site. In vitro splicing ³²P labeled MRJ pre-mRNA was performed in HeLa cell nuclear extract. Antisense morpholino (MoMRJ) or the negative control morpholino (MoC) was added in the reactions (mock, without morpholino). Pre-mRNA and splicing intermediates and products were depicted to the right of the gel.

FIG. 1B shows the RT-PCR and immunoblotting results on RNA and protein levels of MRJ isoforms of HEK293T cells treated with different amounts of MoMRJ or the control MoC in the serum-free medium for 24 h. Bar graphs show relative ratios of MRJ-L to total MRJ (T); data were both obtained from three independent experiments. Asterisks: *p≤0.05; **p≤0.01, ***p≤0.001.

FIG. 2A shows the RT-PCR and immunoblotting results on RNA and protein levels of MRJ isoforms of THP-1 cells cultured in the presence of 160 nM PMA for 24 h to differentiate into macrophages, followed by treatment with MoMRJ or the control MoC in the serum free medium for 24 h. Asterisks: *p≤0.05 **p≤0.01.

FIG. 2B shows the viral p24 Gag protein in culture supernatants detected by ELISA in THP-1-derived macrophages cultured and treated with morpholinos as in FIG. 2A, followed by infection with wild type HIV-1. Averages of p24 concentration were obtained from two independent experiments. Asterisks: **p≤0.01.

FIG. 2C shows percentages of HSA representing HIV-1 positive cells, obtained from two independent experiments, in cells cultured and treated as in pane FIG. 2B, followed by infection with murine heat stable antigen CD24 (HSA) of VSV-G pseudotype HIV-1 NL4-3. Percentages of HAS were obtained by FACS analysis using PE-labeled HSA antibody. Asterisks: *p≤0.05.

FIG. 3A shows the RT-PCR and immunoblotting results on RNA and protein levels of MRJ isoforms and their respective controls, actin and GAPDH, of Hep2 cells treated with control MoC or MoMRJ at indicted concentrations in the serum free medium for 24 h. Bar graphs show relative ratios of MRJ-L to total MRJ (T). Asterisks: *p≤0.05; **p≤0.01.

FIG. 3B shows immunoblotting results of RSV F, MRJ isoforms and GAPDH in Hep2 cells treated with morpholino for 48 h followed by infection with RSV A2 strain at MOI 0.1.

FIG. 3C shows the RSV viral titer and RNA levels of the Hep2 cells treated as in FIG. 3B. Viral titer was determined by plaque assays using culture supernatants. RSV RNA level was determined by RT-qPCR of viral nucleoprotein N transcript in the culture supernatants. Asterisks: **p≤0.01, ***p≤0.001.

FIG. 3D shows the relative viral mRNA levels determined by RT-qPCR and normalized with actin in Hep2 cells treated with morpholino for 24 h and subsequently infected with RSV A2 strain at MOI 1 for 12 h. Bar graphs show the averages from three independent experiments. Asterisks: *p≤0.05; **p≤0.01, ***p≤0.001.

### DETAILED DESCRIPTION OF THE EMBODIMENTS

The following specific examples are used to exemplify the present disclosure. A person of ordinary skills in the art can conceive the other advantages of the present disclosure, based on the disclosure of the specification of the present disclosure. The present disclosure can also be implemented or applied as described in different specific examples.

Unless defined otherwise, all technical and scientific terms used herein have the same meaning as commonly understood by those of ordinary skill in the art to which the invention belongs. Although any methods and materials similar or equivalent to those described herein can be used in the practice or testing of the present disclosure, preferred methods and materials are described. For the purposes of the present disclosure, the following terms are defined below.

As used herein, the singular forms “a,” “an” and “the” include plural referents unless the context clearly dictates otherwise. Thus, for example, reference to “an antigen” includes mixtures of antigens; reference to “a pharmaceutically acceptable carrier” includes mixtures of two or more such carriers, and the like. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone).

The present disclosure provides an antiviral agent for inhibition of growth of viruses and thereby treating a disease or condition associated with viral infection. The antiviral agent comprising a nucleotide derivative complementary to MRJ gene, which is used as antisense oligonucleotides, wherein the nucleotide derivative may comprises at least one morpholino nucleotide. Particularly, the nucleotide derivative is complementary to non-coding sequence of the MRJ gene.

By “coding sequence” is meant any nucleic acid sequence that contributes to the code for the polypeptide product of a gene. By contrast, the term “non-coding sequence” refers to any nucleic acid sequence that does not contribute to the code for the polypeptide product of a gene.

The terms “complementary” and “complementarity” refer to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. While perfect complementarity is often desired, some embodiments can include one or more but preferably 6, 5, 4, 3, 2, or 1 mismatches with respect to the target RNA. Variations at any location within the oligomer are included. In certain embodiments, variations in sequence near the termini of an oligomer are generally preferable to variations in the interior, and if present are typically within about 6, 5, 4, 3, 2, or 1 nucleotides of the 5′ and/or 3′ terminus.

The terms “antisense oligomer” or “antisense compound” or “antisense oligonucleotide” or “oligonucleotide” are used interchangeably and refer to a sequence of cyclic subunits, each bearing a base-pairing moiety, linked by intersubunit linkages that allow the base-pairing moieties to hybridize to a target sequence in a nucleic acid (typically an RNA) by Watson-Crick base pairing, to form a nucleic acid:oligomer heteroduplex within the target sequence. The cyclic subunits may be based on ribose or another pentose sugar or, in certain embodiments, a morpholino group (see description of morpholino oligomers below). Also contemplated are peptide nucleic acids (PNAs), locked nucleic acids (LNAs), 2′-0-Methyl oligonucleotides and RNA interference agents (siRNA agents), and other antisense agents known in the art.

Such an antisense oligomer can be designed to block or inhibit translation of mRNA or to inhibit natural pre-mRNA splice processing, or induce degradation of targeted mRNAs, and may be said to be “directed to” or “targeted against” a target sequence with which it hybridizes. In certain embodiments, the target sequence includes a region including an AUG start codon of an mRNA, a 3′ or 5′ splice site of a pre-processed mRNA, a branch point. The target sequence may be within an exon or within an intron. The target sequence for a splice site may include an mRNA sequence having its 5′ end 1 to about 25 base pairs downstream of a normal splice acceptor junction in a preprocessed mRNA. A preferred splice site target sequence is any region of a preprocessed mRNA that includes a splice site or is contained entirely within an exon coding sequence or spans a splice acceptor or donor site. An oligomer is more generally said to be “targeted against” a biologically relevant target, such as a protein, virus, or bacteria, when it is targeted against the nucleic acid of the target in the manner described above.

The terms “morpholino oligomer” or “PMO” (phosphoramidate- or phosphorodiamidate morpholino oligomer) refer to an oligonucleotide analog composed of morpholino subunit structures, where (i) the structures are linked together by phosphorus-containing linkages, one to three atoms long, preferably two atoms long, and preferably uncharged or cationic, joining the morpholino nitrogen of one subunit to a 5′ exocyclic carbon of an adjacent subunit, and (ii) each morpholino ring bears a purine or pyrimidine or an equivalent base-pairing moiety effective to bind, by base specific hydrogen bonding, to a base in a polynucleotide. Variations can be made to this linkage as long as they do not interfere with binding or activity. For example, the oxygen attached to phosphorus may be substituted with sulfur (thiophosphorodiamidate). The 5′ oxygen may be substituted with amino or lower alkyl substituted amino. The pendant nitrogen attached to phosphorus may be unsubstituted, monosubstituted, or disubstituted with (optionally substituted) lower alkyl. See also the discussion of cationic linkages below. The purine or pyrimidine base pairing moiety is typically adenine, cytosine, guanine, uracil, thymine or inosine. The synthesis, structures, and binding characteristics of morpholino oligomers are detailed in U.S. Pat. Nos. 5,698,685, 5,217,866, 5,142,047, 5,034,506, 5,166,315, 5,521,063, and 5,506,337, and PCT Appn. Nos. PCT/US07/11435 (cationic linkages) and PCT Application No. US2008/012804 (improved synthesis), all of which are incorporated herein by reference.

An “effective amount” or “therapeutically effective amount” refers to an amount of therapeutic compound, such as an antisense oligomer administered to a mammalian subject, either as a single dose or as part of a series of doses, which is effective to produce a desired therapeutic effect. For an antisense oligomer, this effect is typically brought about by inhibiting translation or natural splice-processing of a selected target sequence. An “effective amount,” targeted against a virus, also relates to an amount effective to reduce the rate of replication of the infecting virus, and/or viral load, and/or symptoms associated with the viral infection.

A “decrease” in a response may be “statistically significant” as compared to the response produced by no antisense compound or a control composition, and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease, including all integers in between.

The recitations “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, He, Phe, Tyr, Trp, Lys, Arg, His, Asp, Glu, Asn, Gin, Cys and Met) occurs in both sequences to yield the number of matched positions, dividing the number of matched positions by the total number of positions in the window of comparison (i.e., the window size), and multiplying the result by 100 to yield the percentage of sequence identity.

Treatment includes, but is not limited to, administration of, e.g., a pharmaceutical composition, and may be performed either prophylactically, or subsequent to the initiation of a pathologic event or contact with an etiologic agent. Treatment includes any desirable effect on the symptoms or pathology of a disease or condition associated with virus infection. The related term “improved therapeutic outcome” relative to a patient diagnosed as infected with a particular virus, may refer to a slowing or diminution in the growth of virus, or viral load, or detectable symptoms associated with infection by that particular virus.

Hence, the present disclosure provides a method for treating virus infection, by administering one or more antisense oligomers of the present disclosure (e.g., SEQ ID NO. 1, and variants thereof), optionally as part of a pharmaceutical formulation or dosage form, to a subject in need thereof. A “subject,” as used herein, may include any animal that exhibits a symptom, or is at risk for exhibiting a symptom, which can be treated with an antisense compound of the disclosure, such as a subject that has or is at risk for having an virus infection. Suitable subjects (patients) include laboratory animals (such as mouse, rat, rabbit, or guinea pig), farm animals, and domestic animals or pets (such as a cat or dog). Non-human primates and, preferably, human patients, are included.

The antisense oligomer used in the present disclosure is designed to target to mammalian relative of DnaJ, MRJ. MRJ is also named as DNAJB6, human DnaJ/Hsp40 family member B6, and has two alternatively spliced isoforms, namely the large isoform (MRJ-L) and small isoform (MRJ-S) (Hanai and Mashima, 2003). MRJ-L includes 10 exons, encoding a protein of 326 amino acid residues. MRJ-S does not have the last two exons, so that it lacks the carboxyl-terminal 95 residues of MRJ-L but retains a 10-residue sequence from intron 8.

The present disclosure provides an antisense oligomer that targets MRJ splice site and inhibits its intron 8 splicing, thereby decrease the expression of MRJ-L.

In one embodiment, the nucleotide derivative of the antiviral agent of the present disclosure is complementary to 5′ splice site region of intron 8 of the MRJ gene.

Decrease in the mRNA expression and protein production of MRJ-L, as resulted by the use of antisense oligomer provided in the present disclosure, inhibits virus infection, replication and production in cells. In one embodiment, the inhibition of virus infection, replication and production in cells is by the absence of MRJ-L form and therefore the entering of the virus protein into the nucleus of the cell.

In one embodiment, decrease in the mRNA expression and protein production of MRJ-L, as resulted by the use of antisense oligomer provided in the present disclosure, inhibits HIV-1 replication in cells.

In one embodiment, decrease in the mRNA expression and protein production of MRJ-L, as resulted by the use of antisense oligomer provided in the present disclosure, inhibits RSV-1 replication in cells.

In one embodiment, the antiviral agent of the present disclosure is useful for suppressing viral infection and treating a disease or a condition associated with viral infection.

In one embodiment, the antiviral agent of the present disclosure may be used in combination with other antiviral therapy. The examples of the antiviral therapy include, but is not limited to, carbovir, acyclovir, interferon, stavudine, 3′-azido-2′,3′-dideoxy-5-methyl-cytidine (CS-92), β-D-dioxolane nucleosides, and oseltamivir phosphate.

In one embodiment, the antiviral agent of the present disclosure may be used in combination with an antibiotic when the subject has a secondary bacterial infection.

Many examples have been used to illustrate the present disclosure. The examples below should not be taken as a limit to the scope of the disclosure.

### EXAMPLE

### Cell Cultures and Chemicals

Human embryonic kidney 293T cells (HEK293T) were maintained in Dulbecco's Modified Eagle's medium (DMEM; Hyclone) containing 10% fetal bovine serum (FBS). Human epithelial type 2 (Hep2) cells were cultured in DMEM containing Nutrient Mixture F-12 (DMEM/F12; Thermo Fisher Scientific) supplemented with 10% FBS. Human monocytic THP-1 cells were cultured in RPMI1640 (Hyclone) supplemented with 10% FBS. THP-1 cells were differentiated into macrophage-like cells by adding 160 nM phorbol 12-myristate 13-acetate (PMA; P8139, sigma) into the culture medium for 24 h. Transfection using Lipofectamine 2000 (Invitrogen) was performed according to manufacturer's recommendation.

### In Vitro Splicing Assay

Radio-isotope (³²P)-labeled MRJ pre-mRNA was generated by in vitro transcription using EcoRI-linearized pCDNA-MRJ-e89 vector and T7 polymerase (Promega). The procedure for HeLa nuclear extract preparation and in vitro splicing reaction was as described (Tarn and Steitz, 1994). Morpholinos were added as indicated in figure legends. Total RNA was extracted using TRIzol reagent (Invitrogen) and fractionated on 6% denaturing polyacrylamide gels followed by autoradiography.

### Morpholino Treatment

Morpholino oligonucleotides used in this study included MoMRJ (5′-CAGCATCTGCTCCTTACCATTTATT-3′ (SEQ ID NO. 1); Gene Tools, LLC), complementary to the 5′ splice site region of MRJ intron 8, and negative control MoC (5′-CCTCTTACCTCAGTTACAATTTATA-3′ (SEQ ID NO. 2); Gene Tools, LLC). HEK293T, THP-1 and Hep2 cells were treated with morpholinos in the serum-free medium for 24 h.

### HIV Production and Infection

To generate VSV-G pseudotype of HIV-1 NL4-3, 2×10⁶ HEK293T cells were cotransfected with the NL4-3 HSA R⁺E⁻ vector (obtained from NIH AIDS Reagent Program) and packaging vector pMD.G. To determine viral titers, cell culture supernatants were harvested 48 h post-transfection and subjected to ELISA using anti-p24 Gag (PerkinElmer) (He et al., 1995). THP-1-derived macrophages (see above) were treated with morpholinos in the serum-free medium for 24 h, followed by infection with HIV-1 NL4-3 (20 ng p24 per 1×10⁵ cells) for 48 h. Reporter gene expression was determined by FACS analysis using PE-labeled anti-mouse monoclonal antibody against murine CD24 (HSA) (M1/69; affymetrix eBiosciense). HIVADA strain propagation and titration were as previously described (Chiang et al., 2014). THP-1-derived macrophages were treated with morpholino as above, followed by HIVADA infection (20 ng p24 per 1×10⁵ cells) for 6 days.

### RSV Production and Infection

To propagate RSV, Hep2 cells grown to 80% confluence in 6-well plates were infected with the A2 strain and cultured in the 2% FBS-containing DMEM/F12 medium for 3-4 days. Viral titer was determined in the supernatants by using the plaque assay (McKimm-Breschkin, 2004). In brief, diluted virus was added in Hep2 cells in 6-well plates for 2 h incubation. After absorption, cell was washed by PBS and covered with the mixtures of 2% FBS-containing DMEM/F12 medium and 0.3% agarose at 37° C. incubator for 6 days. Knockdown cells were infected with RSV A2 at multiplicity of infection (MOI) of 0.1 for 2 h. After washing unbound virus using PBS, cells were then incubated for 48 h. Cell lysates were subjected to immunoblotting against the envelope fusion protein (F) of RSV. The supernatants were harvested for plaque assay. Additionally, to evaluate the genomic RNA level, supernatant RNA was subjected to reverse transcription with random primers followed by quantitative PCR (Roche) with specific primers for RSV N (Table 1). The expression of NS1, M2-1 and F genes was examined in infected cells by reverse transcription with oligo(dT) primers and followed by quantitative PCR (Roche) with specific primers (Table 1). For morpholino treatment, cells were absorbed with RSV A2 at MOI of 0.1 for 2 h. After removal of unbound viruses, incubation was continued for another 48 h in the present of morpholinos. Cell lysates and supernatants were collected for analysis as above. Cells were treated with morpholino for 24 h in the serum free medium, and then infected with RSV A2 at MOI 1 for 12 h. Cell lysates were assayed for viral mRNA expression.

### PCR and RT-PCR

Total RNA was extracted using TRIzol reagent (Invitrogen) and subjected to reverse transcription using random primers or oligo(dT) and SuperScript III (Invitrogen) followed by PCR using gene specific primers (Table 1). PCR products were separated on 2% agarose gels.

### Immunoblotting Analysis

Immunoblotting was performed as previously described (Chiang et al., 2014) using an enhanced chemiluminescence detection kit (Thermo Scientific). Antibodies used were against the following proteins or epitopes: MRJ (Abnova, H00010049-A01), RSV F (Santa Cruz Biotech, sc-101362), HA (Convance, 16B12), GFP (Santa Cruz Biotech, sc-8334), and GAPDH (Proteintech, 10494-1-AP). HRP-conjugated secondary antibodies included anti-mouse IgG (SeraCare, 5210-0183) and anti-rabbit IgG (GeneTex, GTX213110-01)

### Statistical Analysis

The GraphPad Prism 5 two-tailed student t test was used to reveal the significance of the experiments. The ImageJ software (National Institutes of Health, USA) was used to quantify bands.

TABLE 1
Primer sets for qPCR and PCR

| | | | SEQ |
|---|---|---|---|
| | Direc- | | ID |
| Gene | tion | Primer sequence | NO. |
| MRJ | Forward | CACTTGATGGCTTACCCTTATG | 3 |
| | | ATGTGCCAGATTATGC | |
| | Reverse | CACTTGGAATTCACCTGCTGCG | 4 |
| | (L form) | GACGCGAGGG | |
| | Reverse | CACTTGGAGCTCGTTCCTGTTA | 5 |
| | (S form) | ATCCTCAAATG | |
| Actin | Forward | AGCACGGCATCGTCACCAAC | 6 |
| | Reverse | TGGCTGGGGTGTTGAAGGTC | 7 |
| Nucleoprotein | Forward | GCAGGATTGTTTATGAATGCC | 8 |
| (N) (RSV A2 | Reverse | CTTCCACAACTTGYTCCATTTC | 9 |
| virus) | | | |
| Fusion protein | Forward | TAAGCAGCTCCGTTATCACATC | 10 |
| (F) (RSV A2 | | TC | |
| virus) | Reverse | ATTGGATGCTGTACATTTAGTT | 11 |
| | | TTGC | |
| M2-1 | Forward | CATGAGCAAACTCCTCACTGAA | 12 |
| (RSV A2 virus) | | CT | |
| | Reverse | TCTTGGGTGAATTTAGCTCTTC | 13 |
| | | ATT | |
| NS1 | Forward | CACAACAATGCCAGTGCTACAA | 14 |
| (RSV A2 virus) | Reverse | TTAGACCATTAGGTTGAGAGCA | 15 |
| | | ATGT | |

### Example 1: Morpholino Oligonucleotide Modulates MRJ Splicing

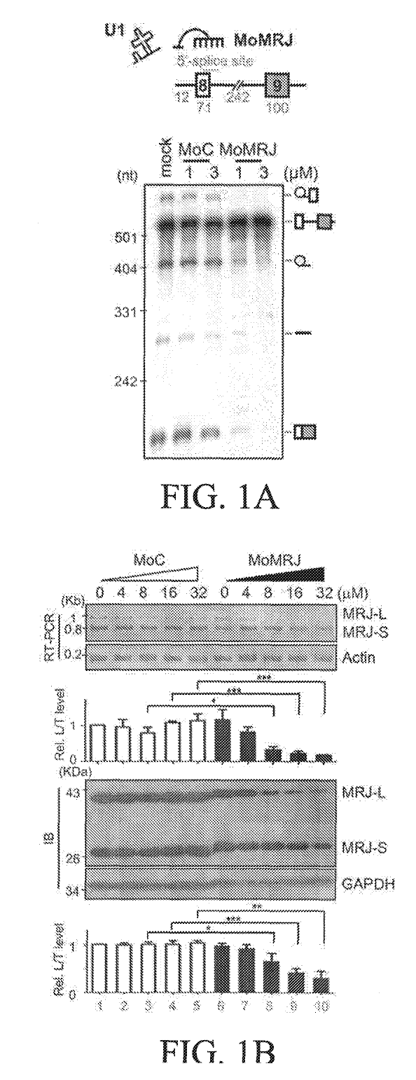

An antisense morpholino oligonucleotide (MoMRJ) having the sequence as indicated in SEQ ID NO. 1 is complementary to the 5′ splice site of intron 8 and was used to interfere with the splicing of the MRJ gene. The efficacy of this morpholino was evaluated with in vitro splicing assay. The MRJ pre-mRNA contained exons 8-9 with an internally truncated intron (FIG. 1A, upper panel). The MRJ pre-mRNA was spliced in the HeLa nuclear extract. MoMRJ inhibited splicing whereas the negative control of morpholino (MoC) had no effect (FIG. 1A, lower panel). This result indicated that MoMRJ specifically disturbed intron 8 splicing. Next, the effect of MoMRJ on MRJ isoform expression in HEK293T cells was assessed. RT-PCR and immunoblotting showed that increasing the amounts of MoMRJ inhibited the inclusion of exons 9/10, thus reducing the expression of MRJ-L mRNA and protein (FIG. 1B, lanes 7-10). MoC did not affect the MRJ ratio (lanes 2-5). Thus, the MRJ splice site targeting morpholino used herein interfered with the expression and amount of MRJ-L in cells.

### Example 2: The MRJ Targeting Morpholino Inhibits HIV-1 Replication

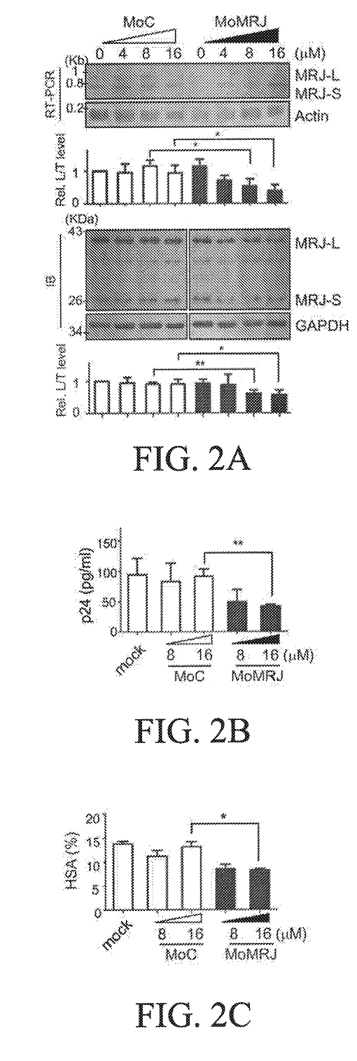

MoMRJ, through its interference with MRJ spicing and suppression of MRJ-L expression, was able to suppress HIV-1 replication in macrophages. As observed in HEK293T cells, MoMRJ, but not MoC, reduced MRJ-L mRNA and protein expression in THP-1 cells (FIG. 2A). MoMRJ was used to treat HIV-1 infected macrophages that were derived from THP-1 (Konopka and Duzgunes, 2002) and evaluated the expression of the HIV core protein p24. The immunosorbent assay revealed that MoMRJ, but not MoC, considerably reduced the level of p24 in the supernatants of HIV-1 infected cells (FIG. 2B). The effect of MoMRJ in the early stage of HIV-1 infection was further assessed using a one-round infection system, in which the VSV-G-pseudotyped HIV-1 NL4-3 strain containing the murine heat-stable antigen CD24 (HSA) gene in the nef region as the reporter (He et al., 1995). The HSA-positive cells were evaluated using fluorescence-activated cell sorting (FACS). As shown in FIG. 2C, MoMRJ could reduce the number of HSA presenting cells, whereas MoC had no significant effect. These results indicated that the MRJ targeting morpholino, by reducing MRJ-L mRNA expression and protein production, inhibited HIV-1 life cycle during the early stage.

### Example 3: The MRJ Targeting Morpholino Inhibits RSV Replication

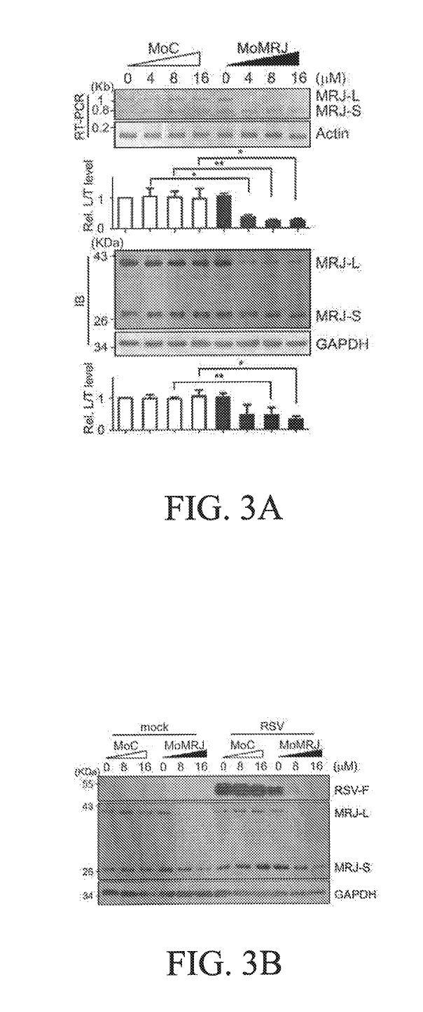

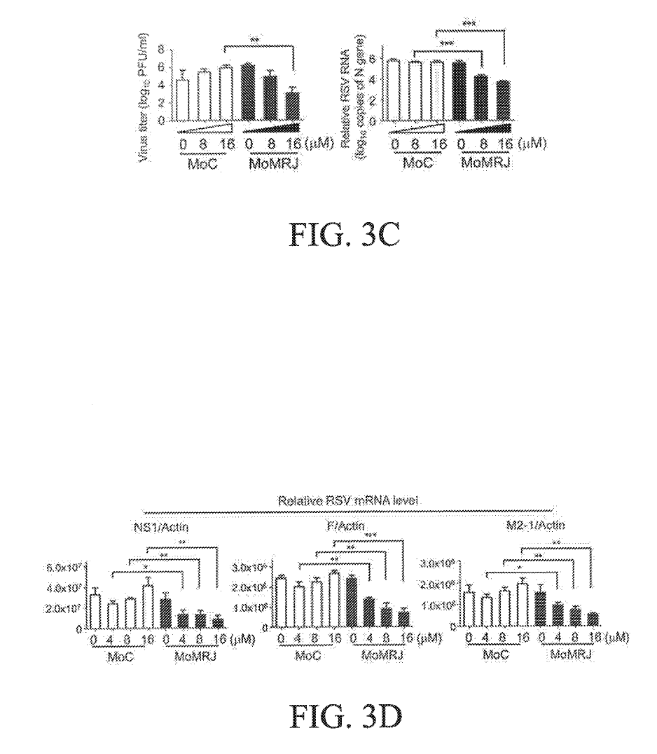

MoMRJ was further used to examine its ability in constraining RSV production. MoMRJ and the control MoC were titrated and used in Hep2 cells. RT-PCR and immunoblotting analysis showed that MoMRJ efficiently reduced the mRNA and protein levels of MRJ-L but not MRJ-S (FIG. 3A). Levels of RSV infection in morpholino treated cells were then evaluated. Immunoblotting showed that RSV F protein expression was drastically down-regulated in MoMRJ-treated cells (FIG. 3B). Plaque assay and RT-qPCR of RSV N mRNA confirmed that MoMRJ substantially suppressed virion production (FIG. 3C). Viral subgenomic mRNAs production were also reduced upon MoMRJ treatment while MoC showed no effect (FIG. 3D). Thus, MoMRJ showed RSV-inhibiting effect by reducing mRNA and protein levels of MRJ-L.

What is claimed is:

1. An antiviral agent comprising a nucleotide derivative complementary to a mammalian relative of DnaJ (MRJ) gene, wherein the nucleotide derivative comprises at least one nucleotide with a sugar moiety being substituted with morpholine.

2. The antiviral agent according to claim 1, wherein the nucleotide derivative is complementary to intron 8 of the MRJ gene.

3. The antiviral agent according to claim 1, wherein the nucleotides in the nucleotide derivative are morpholino nucleotides.

4. The antiviral agent according to claim 1, wherein the nucleotide derivative comprises SEQ ID NO: 1.

5. The antiviral agent according to claim 1, wherein the nucleotide derivative consists of a sequence of SEQ ID NO: 1.

6. The antiviral agent of claim 1 for use in treating a disease or a condition associated with viral infection in a subject in need thereof.

7. The antiviral agent according to claim 6, wherein the viral infection is caused by a virus selected from the group consisting of cytomegalovirus (CMV), Epstein-Barr virus (EBV), human immunodeficiency virus-1 (HIV-1), human immunodeficiency virus-2 (HIV-2), human metapneumovirus, human parainfluenza virus (HPIV), influenza virus, respiratory syncytial virus (RSV), adenovirus, rhinovirus, coronavirus, enterovirus 71 (EV-71), Enterovirus D68 (EV-D68), coxsackievirus, dengue virus, Japanese encephalitis virus (JEV), and any combination thereof.

8. The antiviral agent according to claim 7, wherein the viral infection is caused by CMV, EBV, HIV, influenza virus, RSV or any combination thereof.

9. The antiviral agent according to claim 8, wherein the viral infection is caused by RSV.

10. The antiviral agent according to claim 6, wherein the disease or the condition is selected from the group consisting of retinitis, colitis, infectious mononucleosis, Hodgkin's lymphoma, Burkitt's lymphoma, nasopharyngeal carcinoma, acquired immune deficiency syndrome (AIDS), lower respiratory tract infection (LRI), myocarditis, encephalitis, dengue hemorrhagic fever and dengue shock syndrome (DHF/DSS), and any combination thereof.

11. A method for suppressing viral infection, comprising administering the antiviral agent of claim 1 to a subject in need thereof.

12. The method according to claim 11, wherein the viral infection is caused by a virus selected from the group consisting of cytomegalovirus (CMV), Epstein-Barr virus (EBV), human immunodeficiency virus-1 (HIV-1), human immunodeficiency virus-2 (HIV-2), human metapneumovirus, human parainfluenza virus (HPIV), influenza virus, respiratory syncytial virus (RSV), adenovirus, rhinovirus, coronavirus, enterovirus 71 (EV-71), Enterovirus D68 (EV-D68), coxsackievirus, dengue virus, Japanese encephalitis virus (JEV), and any combination thereof.

13. The method according to claim 11, wherein the viral infection is caused by RSV.

14. The method according to claim 11, further comprising administering an additional antiviral therapy to the subject.

15. The method according to claim 14, wherein the additional antiviral therapy is selected from the group consisting of carbovir, acyclovir, interferon, stavudine, 3′-azido-2′,3′-dideoxy-5-methyl-cytidine (CS-92), β-D-dioxolane nucleosides, oseltamivir phosphate, and any combination thereof.
